(19) United States
(12) Patent Application Publication
(10) Pub. No.: US 2025/0228678 A1
(43) Pub. Date: Jul. 17, 2025

(54) DELIVERY SYSTEMS FOR MULTIPLE ENDOANCHORS

(71) Applicant: Medtronic Vascular, Inc., Santa Rosa, CA (US)

(72) Inventors: David E. FLORY, Santa Rosa, CA (US); Anish S. NIGADE, Santa Rosa, CA (US); Antonia TROTTA, Galway (IE)

(21) Appl. No.: 18/989,190

(22) Filed: Dec. 20, 2024

Related U.S. Application Data
(60) Provisional application No. 63/619,967, filed on Jan. 11, 2024.

(51) Int. Cl. A61F 2/95

(52) U.S. Cl. CPC A61F 2/95; A61F 2220/0016

(57) ABSTRACT

An endoanchor delivery system includes an endoanchor sheath defining an endoanchor sheath lumen. The endoanchor sheath defines a side opening within the distal portion of the endoanchor sheath. The endoanchor delivery system includes a series of endoanchors loaded into the lumen at a distal portion thereof and disposed along a longitudinal axis of the applier catheter in a loaded state. The series of endoanchors includes a first endoanchor and a second endoanchor. The first endoanchor is located inward the second endoanchor. The endoanchor delivery system further includes an applier catheter extendible within the endoanchor sheath. The applier catheter is configured to load the first endoanchor at a distal portion of the applier catheter and to extend the distal portion of the applier catheter through the side opening to deploy the first endoanchor to anchor an implant to a blood vessel wall.

### CROSS-REFERENCE TO RELATED APPLICATIONS

This application claims the benefit of U.S. Provisional Patent Application Ser. No. 63/619,967, filed Jan. 11, 2024, the entire content of which is incorporated herein by reference.

### TECHNICAL FIELD

The present disclosure relates to delivery systems for multiple endoanchors.

### BACKGROUND

An aortic aneurysm may be an enlarged area in an aorta (e.g., an abdominal aorta). The aortic aneurysm may form from the degradation of elastin and/or interstitial collagen, which may alter the structural integrity of the aortic wall, which may weaken it. One current treatment of an aortic aneurysm may be endovascular surgery which utilizes an implant (e.g., stent graft). Another more invasive option is open surgery.

Endovascular procedures are minimally invasive techniques to deliver clinical treatments in a patient's vasculature (e.g., treatment of aortic aneurysms). One example of a clinical treatment used in an endovascular procedure is deployment of a stent graft. A conventional stent graft typically includes a radially expandable reinforcement structure, e.g., formed from a plurality of annular stent rings, and a cylindrically shaped layer of graft material defining a lumen to which the stent rings are coupled. The stent graft is placed inside a patient's vasculature (e.g., blood vessel) to bridge a diseased blood vessel segment (e.g., an aneurismal, dissected, or torn blood vessel segment), and thereby excluding hemodynamic pressures of blood flow from the diseased blood vessel segment.

The proximal end of the stent graft is expended onto a landing zone of the diseased blood vessel. The anatomy of the landing zone may compromise the fixation and sealing of the stent graft, thereby potentially causing leaking and/or movement of the stent graft. Staples may be applied to the proximal end of the stent graft to anchor the stent graft to the blood vessel wall.

### SUMMARY

In an embodiment, an endoanchor delivery system is disclosed. The endoanchor delivery system includes an endoanchor sheath defining an endoanchor sheath lumen. The endoanchor sheath has a proximal portion and a distal portion. The endoanchor sheath defines a side opening within the distal portion of the endoanchor sheath. The endoanchor delivery system includes a series of endoanchors loaded into the lumen at the distal portion thereof and disposed along a longitudinal axis of the endoanchor sheath in a loaded state. The series of endoanchors includes a first endoanchor and a second endoanchor. The first endoanchor is located inward the second endoanchor along the longitudinal axis of the applier catheter. The endoanchor delivery system further includes an applier catheter extendible within the endoanchor sheath. The applier catheter has a proximal portion and a distal portion. The applier catheter is configured to load the first endoanchor at the distal portion of the applier catheter and to extend the distal portion of the applier catheter through the side opening to deploy the first endoanchor to anchor an implant to a blood vessel wall.

The endoanchor delivery system may further include an endoanchor cartridge located in the distal portion of the endoanchor sheath. The endoanchor cartridge may be configured to carry a series of endoanchors. The endoanchor cartridge may include a first flap secured proximal the first endoanchor and a second flap secured proximal the second endoanchor. The first flap has a holding state to hold the first endoanchor within the endoanchor cartridge and a loading state to load the first endoanchor to the applier catheter. The second flap has a holding state to hold the second endoanchor within the endoanchor cartridge and a loading state to load the first endoanchor to the applier catheter. The first and/or second flaps may be multi-faceted flaps including circumferentially arranged tapered flap facets. The endoanchor cartridge may include a first support system configured to releasably secure the first endoanchor and a second support system configured to releasably secure the second endoanchor. The first support system may include one or more areas of weakness and the second support system may include one or more areas of weakness.

In one or more embodiments, the endoanchor sheath includes an inner surface and an outer surface. The endoanchor delivery system may further include a biasing element secured to the inner surface of the sheath. The biasing element at least partially aligning with the side opening. The biasing element may include a stretched state during loading of the first endoanchor by the applier catheter and an unstretched state when deploying the first endoanchor through the side opening. The biasing element may include a distal base portion, a proximal base portion, and a middle portion extending therebetween. The distal base may be fixedly secured to the inner surface of the endoanchor sheath, and the proximal base may be slidably secured to the inner surface of the sheath. The endoanchor delivery system may further include a tether secured to the proximal base portion and configured to transition the biasing element from the stretched state to the unstretched state. In one or more embodiments, the middle portion may bow outward in the unstretched state to create a stop to deflect the applier catheter through the side opening.

In one or more embodiments, the endoanchor delivery system includes an endoanchor fixture extending from the distal portion of the applier catheter an extending length corresponding to a length of the first endoanchor. The endoanchor fixture may be configured to releasably hold the first endoanchor to withdraw it proximally from the endoanchor cartridge. The endoanchor delivery system may further include a tapered tip extending from the distal portion of the endoanchor sheath.

In another embodiment, an endoanchor delivery system is disclosed. The endoanchor delivery system includes an endoanchor sheath having a proximal portion terminating in a proximal end and a distal portion terminating in a distal end. The endoanchor sheath defines an endoanchor sheath lumen. The endoanchor sheath defines a side opening within the distal portion of the endoanchor sheath. The endoanchor cartridge is located within the distal portion of the endoanchor sheath and extends along a longitudinal axis. The endoanchor delivery system may further include a series of endoanchors loaded into the endoanchor cartridge and disposed along the longitudinal axis of the endoanchor cartridge in a loaded state. The series of endoanchors includes a first endoanchor and a second endoanchor. The first endoanchor is located inward the second endoanchor along the longitudinal axis of the endoanchor cartridge. The endoanchor delivery system further includes an applier catheter extendible within the endoanchor sheath. The applier catheter has a proximal portion and a distal portion. The endoanchor delivery system also includes an endoanchor dispenser extending from the distal end of the endoanchor sheath. The endoanchor dispenser is configured to be actuated to load the first endoanchor at the distal portion of the applier catheter. The applier catheter is configured to extend the distal portion of the applier catheter through the side opening to deploy the first endoanchor to anchor an implant to a blood vessel wall.

The endoanchor cartridge may include a helical track to retain the first endoanchor and the second endoanchor. The endoanchor dispenser may be configured to rotate the helical track to urge the first endoanchor to axially translate from a distal position to a proximal position within the endoanchor cartridge to load the first endoanchor to the applier catheter. The endoanchor dispenser may include a cam body, a plunger, and a stop member collectively configured to load the first endoanchor to the applier catheter. The stop member may be fixed to the endoanchor sheath. The cam body may be configured to rotate and axially translate distally and proximally at least partially within the endoanchor dispenser. The plunger may be configured to axially translate distally and proximally at least partially within the endoanchor dispenser.

In yet another embodiment, an endoanchor delivery system is disclosed. The endoanchor delivery system includes a shaft extending to define a shaft lumen. The has a proximal portion and a distal portion. The shaft includes an inner surface has an internal threading and an outer surface. The internal threading is adjacent a distal end of the shaft. The endoanchor delivery system includes a series of endoanchors loaded into the shaft lumen at the distal portion thereof and disposed along a longitudinal axis of the shaft. The series of endoanchors includes a first endoanchor and a second endoanchor. The first endoanchor is located inward the second endoanchor along the longitudinal axis of the shaft. The endoanchor delivery system includes a driver extending along the longitudinal axis of the shaft and through the inner diameters of the first and second endoanchors. The endoanchor delivery system further includes a spring situated with the shaft. A combination of the spring imparting a spring force and the driver rotating collectively loads the second endoanchor into the internal threading.

The endoanchor delivery system may further include an actuator configured to actuate the spring force and the driver rotation via a first actuation of the actuator. The first actuation is configured to translate the second endoanchor into the internal threading. The actuator may be configured to actuate the spring force and the driver rotation via a second actuation of the actuator. The second actuation is configured to translate the first endoanchor into the internal threading and the second endoanchor into a deployment position within an implant and a blood vessel wall.

In one or more embodiments, the internal threading has a distal threading length, and the second endoanchor has a second endoanchor length complimentary to the distal threading length.

### BRIEF DESCRIPTION OF THE DRAWINGS

FIG. 1A is a perspective view of a prior art endoanchor delivery system including an endoanchor guide and an endoanchor applier.

FIG. 1B is a perspective view of a prior art endoanchor cassette defining apertures for receiving endoanchors.

FIG. 1C is a cross section view of an abdominal aorta and a side view of prior art endoanchor catheter in a deployment state deploying an endoanchor to anchor a stent graft to a wall of the abdominal aorta.

FIG. 1D is a magnified view of a distal portion of the endoanchor guide in a deployment state deploying the endoanchor shown in FIG. 1C.

FIG. 2A is a cross section view of an endoanchor delivery system having an endoanchor cartridge.

FIG. 2B shows a magnified view of a portion of the sheath of the endoanchor delivery system showing a biasing element in a stretched state.

FIG. 2C shows a magnified view of a portion of the sheath of the endovascular delivery system showing the biasing element in an unstretched state.

FIG. 2D is a cross section view of a proximal portion of the endoanchor cartridge including a first flap and a second flap at a proximal end thereof and configured to open unidirectionally in a distal direction.

FIG. 2E depicts a cross section view of the endoanchor cartridge including endoanchors contained by multi-faceted flaps.

FIG. 2F shows a bottom view of an exemplar multi-faceted flap including circumferentially arranged tapered flap facets forming a central opening.

FIG. 2G depicts a cross section view of a portion of the endoanchor cartridge showing a multi-faceted flap partially extending within an endoanchor.

FIG. 2H depicts an endoanchor fixture extending from the distal end of the applier catheter into an internal cavity of the endoanchor.

FIG. 2I depicts peripheral support systems secured to the endoanchor cartridge for releasably securing endoanchors according to another embodiment.

FIG. 2J is a magnified, isolated view of an endoanchor and a second lower support.

FIG. 3A depicts a schematic, cross section view of a portion of an endoanchor delivery system including an endoanchor cartridge and an endoanchor dispenser according to one embodiment.

FIG. 3B depicts a schematic, cross section view of the endoanchor cartridge including a helical track and protrusions extending from the helical track.

FIGS. 3C, 3D, and 3E depict perspective views of a cam body, a plunger, and first and second stop members of the endoanchor dispenser according to one embodiment.

FIG. 3F depicts a schematic side view of the endoanchor dispenser in a first deployment position between a retracted position and a loaded position.

FIG. 3G depicts a schematic side view of the endoanchor dispenser in a second deployment position moving toward the loaded position.

FIG. 3H depicts an endoanchor dispensing system in the loaded position.

FIG. 4 depicts a cutaway side view of a distal region of an endoanchor applier.

FIG. 5A depicts a side, schematic view of an endoanchor delivery system including a sheath and an applier catheter extending within the sheath.

FIG. 5B depicts a side, schematic, isolated view of the sheath shown in FIG. 5A.

FIG. 5C depicts a side, schematic, isolated view of the applier catheter shown in FIG. 5A.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

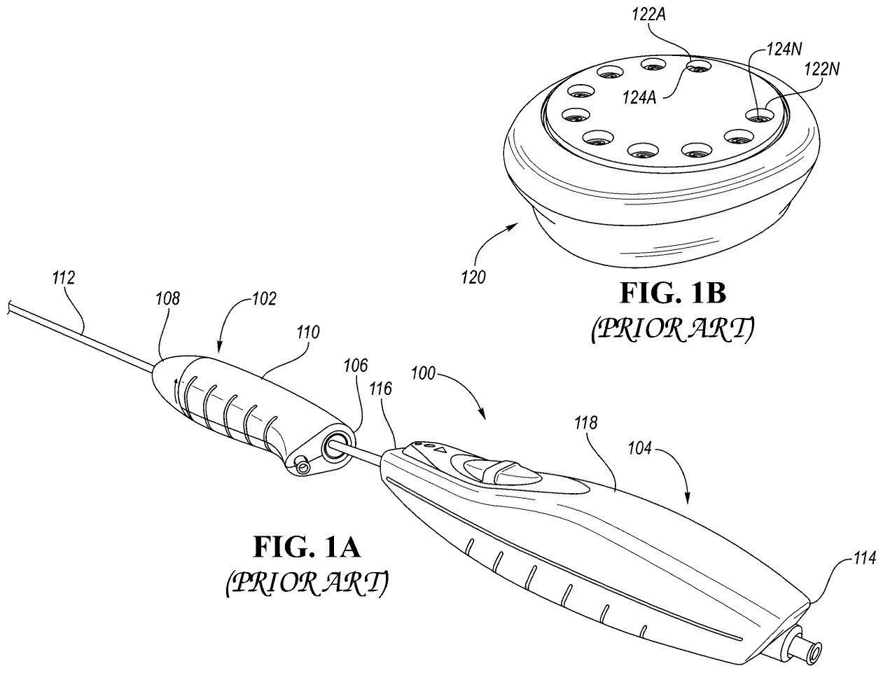

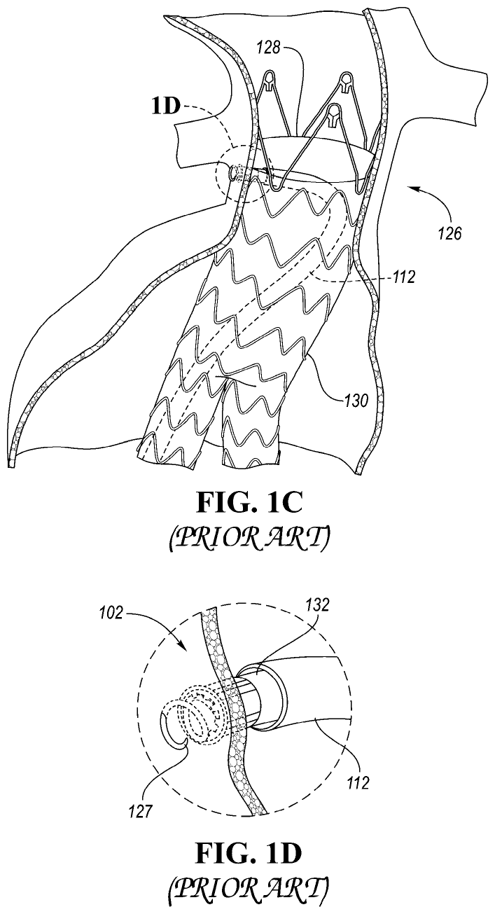

FIG. 1A is a perspective view of prior art endoanchor delivery system 100 including endoanchor guide 102 and endoanchor applier 104. Endoanchor guide 102 includes proximal end 106 and distal end 108 and body 110 extending therebetween. Endoanchor sheath 112 extends from distal end 108 of endoanchor guide 102. Endoanchor applier 104 include proximal end 114 and distal end 116 and body 118 extending therebetween.

FIG. 1B is a perspective view of prior art endoanchor cassette 120 defining apertures 122A through 122N for receiving endoanchors 124A through 124N, respectively. One of endoanchors 124A through 124N is loaded into a distal end of endoanchor guide 102. Endoanchor delivery system 100 is configured to deliver the loaded endoanchor to a delivery site to provide fixation and sealing between an endovascular implant (e.g., an endovascular stent graft) and a wall of native blood vessel (e.g., a thoracic aorta or an abdominal aorta). Endoanchor delivery system 100 is withdrawn from the vasculature of a patient after deployment of the loaded endoanchor so that a subsequent endoanchor may be loaded into the distal end of endoanchor guide 102.

FIG. 1C is a cross section view of abdominal aorta 126 and a side view of endoanchor sheath 112 in a deployment state deploying loaded prior art endoanchor 127 to anchor proximal end 128 of stent graft 130 to a wall of abdominal aorta 126. FIG. 1D is a magnified view of a distal portion of prior art endoanchor guide 102 in a deployment state deploying loaded endoanchor 127. The distal portion of endoanchor guide 102 includes endoanchor sheath 112 and endoanchor applier 132. As shown in FIG. 1C, endoanchor applier 132 is curved by deflectable endoanchor sheath 112, such that endoanchor applier 132 and endoanchor 127 are apposed against stent graft 130. Deflectable endoanchor sheath 112 may deflect its tip up to about 90 degrees or more relative to its undeflected, straight configuration. Distal portion of endoanchor applier 132 is about 90 degrees relative to stent graft 130 such that endoanchor applier 132 reaches stent graft 130.

Endoanchor delivery system 100 is configured to load one endoanchor 127 at a time. Endoanchor delivery system 100 is removed from a patient's vasculature after each endoanchor 127 is delivered. The single delivery of endoanchors 127 increases the procedural time to anchor an implant to a blood vessel wall. Also, orienting endoanchor applier 132 in three-dimensional spaces such that endoanchor applier 132 and endoanchor 127 is apposed against stent graft 130 is challenging. During case planning before a procedure, a rotation angle reference of endoanchor applier 132 may be determined to help meet this challenge. Orientation of endoanchor applier 132 during the procedure may also aid in successful endoanchor placement. However, the patient's anatomy may cause further challenged. For instance, the acute angulation in the abdominal aorta and/or the overall aorta size may make deflection of endoanchor sheath 112 and proper orientation challenging. This may result in inadequate wall penetration or loss of endoanchor 127.

In light of the foregoing, what is needed is an endoanchor delivery system in which multiple (e.g., two or more endoanchors) endoanchors may be loaded into the same applier catheter where the endoanchors may be deployed with proper positioning successively during one procedure (e.g., without removing the applier from the patient's anatomy to reload subsequent endoanchors).

In one or more embodiments, an endoanchor delivery system is disclosed. The endoanchor delivery system includes an endoanchor guide and an endoanchor applier. The endoanchor guide and the endoanchor applier may be part of an endoanchor handle system. The endoanchor applier includes an applier catheter configured to load multiple (e.g., two or more endoanchors) endoanchors for deployment successively during one procedure (e.g., without removing from the patient to reload). The endoanchor guide and/or applier may be deflectable, such that the tip may have an angle of 90 degrees or less relative to the longitudinal axis (e.g., perpendicular or acute). This system and the related method have one or more benefits (e.g., reduced procedure time, increased accuracy of positioning of one or more of the multiple endoanchors, reduced risk associated with removing and re-inserting endoanchor delivery device into patient, and/or permit treatment of patient's with more acute vessel angulation).

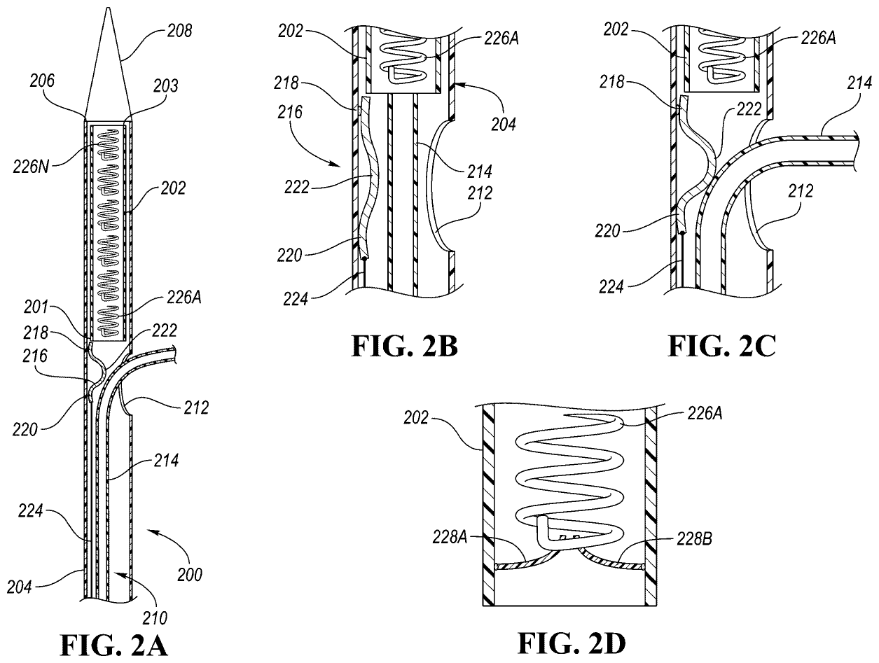

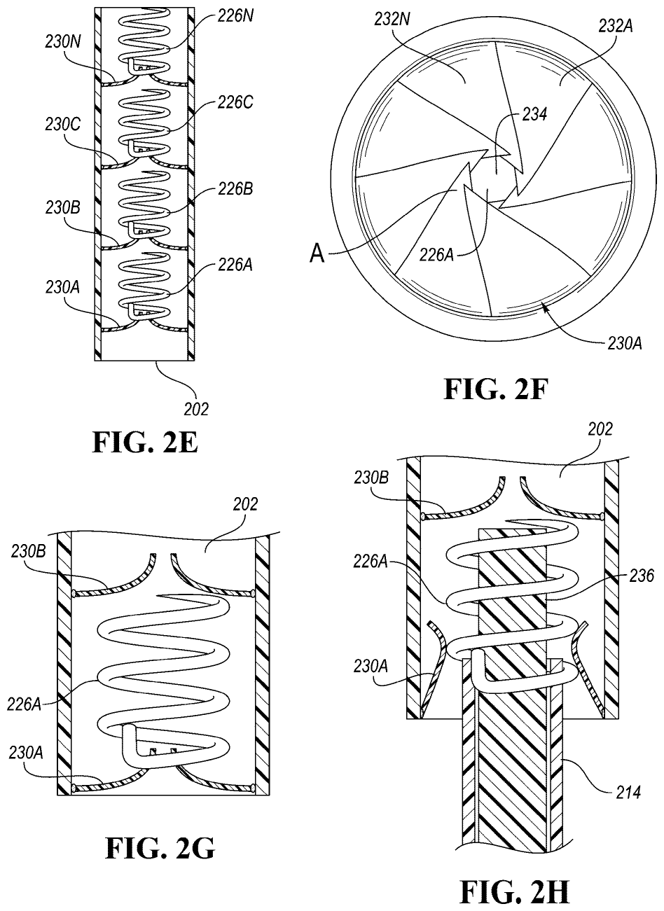

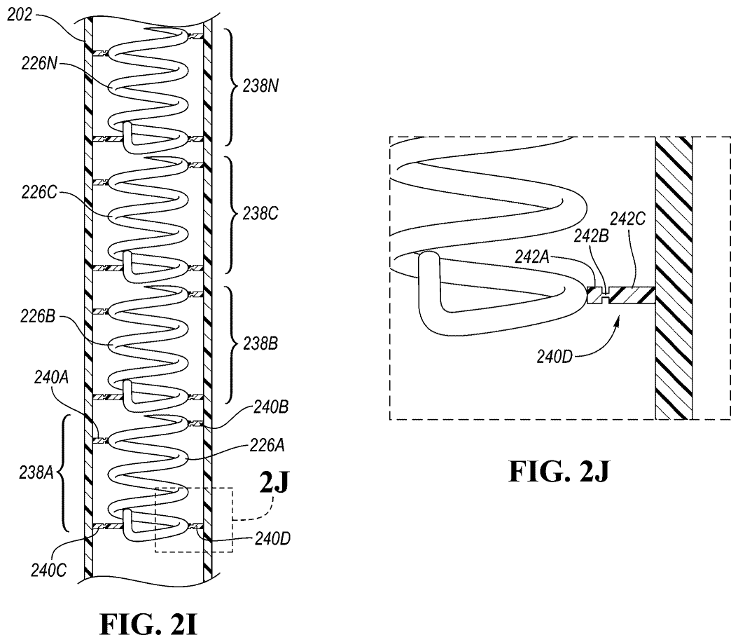

FIG. 2A is a cross section view of endoanchor delivery system 200 having endoanchor cartridge 202. Endoanchor cartridge 202 includes proximal end 201 and distal end 203. Distal end 203 of endoanchor cartridge 202 may be secured to endoanchor delivery system 200 includes endoanchor sheath 204 having distal end 206 and tapered tip 208 extending from distal end 206 of sheath 204. As shown in FIG. 2A, tip 208 has a cone shape that narrows from a wide base adjacent to distal end 206 of sheath 204 toward a narrow end (e.g., a point). The shape of tip 208 is configured to guide endoanchor delivery system 200, including endoanchor sheath 204, through a patient's vasculature and to a deployment site for endoanchors included in endoanchor delivery system 200 (e.g., over a guidewire). Distal end 203 of endoanchor cartridge 202 may be secured to the wide base of tip 208 at distal end 206 of sheath 204.

Endoanchor sheath 204 defines endoanchor sheath lumen 210 and side opening 212. Applier catheter 214 is configured to extend within lumen 210 of sheath 204 and is configured to deflect so that applier catheter 214 can extend through side opening 212. In one or more embodiments, applier catheter 214 extends proximally to connect with a handle (not shown) of endoanchor delivery system 200. Biasing element 216 is secured to an inner surface of sheath 204. Biasing element 216 is located adjacent proximal end 201 of endoanchor cartridge 202. Biasing element 216 includes distal base portion 218 and proximal base portion 220 secured to the inner surface of sheath 204 and middle portion 222 extending between distal base portion 218 and proximal base portion 220. Tether 224 is secured to proximal base portion 220 of biasing element 216 and extends proximally therefrom to the handle, which is configured to manipulate tether 224 to transition biasing element 216 from a stretched state to an unstretched state.

Endoanchor cartridge 202 is configured to receive, contain, and dispense a series of endoanchors 226A through 226N, which are situated within endoanchor cartridge 202 in a stacked arrangement. Endoanchors 226A through 226N have a helical shape, but endoanchor cartridge 202 may contain endoanchors having different shapes (e.g., curved shape) suitable to anchor an implant (e.g., stent graft) to a blood vessel wall. While FIG. 2A depicts six (6) endoanchors housed within endoanchor cartridge 202, the length of the cartridge may be modified to house less or more endoanchors (e.g., 2, 3, 4, 5, 6, 7, 8, 9, 10, 11, 12, 13, 14, 15, 16, 17, 18, 19, and 20 or any range of these two values).

FIG. 2B is a magnified view of a portion of sheath 204 showing biasing element 216 in the stretched configuration. When biasing element 216 is in the stretched state, applier catheter 214 may be inserted into endoanchor cartridge 202 to load an endoanchor onto the distal end of applier catheter 214 or into a lumen defined by applier catheter 214. Biasing element 216 may be translated into the stretched state by retracting tether 224 (e.g., retracting it with the handle of endoanchor delivery system 200). Distal base portion 218 is fixedly secured to the inner surface of sheath 204 such that distal base portion 218 does not move when tether 224 is retracted. Proximal base 220 is slidably secured to the inner surface of sheath 204 such that proximal base 220 moves proximally when tether 224 is retracted while not separating from the inner surface of sheath 204. Tether 224 is fixedly secured to proximal base portion 220 such that the proximal movement of tether 224 is translated to proximal base portion 220 to move biasing element 216 into the stretched state. Applier catheter 214 may extend into endoanchor cartridge 202 in a straight state without deflecting on biasing element 216 in the stretched state.

FIG. 2C shows a magnified view of a portion of sheath 204 of endoanchor delivery system 200 showing biasing element 216 in an unstretched state. When biasing element 216 is in the unstretched state, applier catheter 214 is deflected by biasing element 216 through side opening 212 such that the endoanchor loaded by applier catheter 214 may be delivered to anchor an implant to a blood vessel wall. Biasing element 216 may be translated into the unstretched state by advancing tether 224 (e.g., advancing it with the handle of endoanchor delivery system 200) or releasing tension on the tether 224 and allowing the biasing element 216 to resiliently return to its unstretched state. With distal base portion 218 fixedly secured to the inner surface of sheath 204 and proximal base portion 220 slidably secured to the inner surface of sheath 204, the advancement/release of tether 224 bows out middle portion 222 as shown in FIG. 2C, thereby creating stop for deflecting applier catheter 214 through side opening 212. In one or more embodiments, biasing element 216 may regain its unstretched state when applier catheter 214 is proximally withdrawn past biasing element 216. While the biasing element 216 is shown with the middle portion 222 bowed in the unstretched/natural state and straightened in the stretched state, it may be reversed in other embodiments. For example, the straightened configuration in FIG. 2B may be the natural state and the biasing element 216 may be compressed to transition it to the bowed shape in FIG. 2C. In such embodiments, the tether may be a wire/filament having sufficient compressive strength to buckle the biasing element 216. In embodiments where a tensile force is applied to the tether 222, the material may be a more flexible material such as a suture or any other suitable material.

Biasing element 216 may be formed of a material having a resilient characteristic and a flexible characteristic. The resilient characteristic is configured to deflect applier catheter 214 when biasing element 216 is in the unstretched state. The flexible characteristic is configured to permit biasing element 216 to transition between the unstretched state and the stretched state. Non-limiting examples of materials for biasing element 216 include nitinol or stainless steel. Tether 224 may be formed of a material having a resilient characteristic and a flexible characteristic. The resilient characteristic is configured to push (if necessary) biasing element 216 from the stretched state to the unstretched state and to pull biasing element 216 from the unstretched state to the stretched state. The flexible characteristic is configured to permit tether 224 to bend when travelling through a patient's vasculature to a treatment site. Non-limiting examples of materials for tether 224 include a monofilament or multifilament tether made from nylon, polyester or any other known fabric used as a suture or a stitching material. Tether 224 may also be formed from an alloy such as nitinol wire or stainless steel wire.

FIG. 2D is a cross section view of a proximal portion of endoanchor cartridge 202 including first flap portion 228A and second flap portion 228B at a proximal end thereof. First flap portion 228A and second flap portion 228B, which may be collectively referred to as a flap, are configured to open unidirectionally in a distal direction. First flap portion 228A and second flap portion 228B are anchored at one end thereof to the inner surface of endoanchor cartridge 202 and curve upward from the anchor points to free ends. The free ends of first flap portion 228A and second flap portion 228B are spaced apart. When applier catheter 214 advances, it contacts the sloping sides of first flap portion 228A and second flap portion 228B to further space apart the free ends to permit applier catheter entry into endoanchor cartridge 202 to load endoanchor 226A into or onto the distal end of applier catheter 214. On the other hand, first flap portion 228A and second flap portion 228B are configured to hold endoanchor 226A in place in a holding state such that it cannot otherwise exit endoanchor cartridge 202 in the proximal direction. First flap portion 228A and second flap portion 228B are biased to open in a distal direction to permit loading of applier catheter 214 with endoanchor 226A and are biased to close in a proximal direction to prevent endoanchors (e.g., endoanchor 226A) from escaping endoanchor cartridge 202. Although two (2) flaps are shown, in other embodiments, more than two (2) flaps may be implemented (e.g., 3, 4, 5, 6, 7, 8, 9, and 10 flaps). While a flap is shown only at the proximal end of cartridge 202 in FIG. 2D, flaps may be positioned between each endoanchor 226B to 226N above/distal to endoanchor 226A, as well, to separate the endoanchors. Applier catheter 214 may deflect each flap it encounters as successive endoanchors are loaded and delivered.

FIG. 2E depicts a cross section view of endoanchor cartridge 202 including endoanchors 226A, 226B, 226C, and 226N contained by multi-faceted flaps 230A, 230B, 230C, and 230N. As shown in FIG. 2E, multi-faceted flaps 230A, 230B, 230C, and 230N are sloped upward in a distal direction to contact and to support endoanchors 226A, 226B, 226C, and 226N, respectively. As shown in FIG. 2E, each endoanchor is placed between an adjacent pair of multi-faced flaps to separate the endoanchors from each other for delivery as described herein.

FIG. 2F shows a bottom view of multi-faceted flap 230A including circumferentially arranged tapered flap facets 232A through 232N forming central opening 234. As shown in FIG. 2F, endoanchor 226A is visible through central opening 234. Central opening 234 is configured to open upon a threshold force being applied by applier catheter 214. Central opening 234 may be configured such that it only opens upon the threshold force being applied and stays closed otherwise. In other embodiments, circumferentially arranged tapered flap facets 232A through 232N close onto each other such that there is no central opening.

As shown in FIG. 2F, adjacent pairs of tapered flap facets 232A through 232N overlap each other to reinforce the strength of multi-faceted flap 230A to support endoanchor 226A. While a taper angle A shown in FIG. 2F is about 30 degrees, the taper angle may be greater than 30 degrees to facilitate opening of multi-faceted flap 230A by distal movement of applier catheter 214 while not permitting endoanchor 226A to exit out the proximal end of endoanchor cartridge 202. The taper angle may be any of the following angles or in a range of any two of the following angles: 30, 35, 40, 45, 50, 55, 60, 65, 70, and 75 degrees.

FIG. 2G depicts a cross section view of a portion of endoanchor cartridge 202 showing multi-faceted flap 230A partially extending within endoanchor 226A. As shown in FIG. 2G, endoanchor 226A has a helical shape forming an internal cavity for receiving a portion of multi-faceted flap 230A. As shown in FIG. 2G, the ends of multi-faceted flap 230A extend within a central portion of the internal cavity. The proximal end of endoanchor 226A may rest on central portions of the multi-faceted flap 230A to further support endoanchor 226A.

FIG. 2H depicts endoanchor fixture 236 extending from the distal end of applier catheter 214 into the internal cavity of endoanchor 226A. Endoanchor fixture 236 has a cross sectional shape along its longitudinal axis that is configured to advance within the internal cavity of endoanchor 226A. The cross sectional shape may be a “D” shaped. In one or more embodiments, the length of endoanchor fixture 236 that extends beyond the distal end of applier catheter 214 corresponds to (e.g., similar or the same) the length of endoanchor 226A so that endoanchor fixture 236 is configured to fixate one endoanchor at a time while the other endoanchors stay in place within endoanchor cartridge 202. Endoanchor fixture 236 is configured to releasably hold a single endoanchor so that it can be withdrawn proximally from endoanchor cartridge 202. As shown in FIG. 2H, the distal end of applier catheter 214 pushes apart multi-faceted flap 230A so that endoanchor fixture 236 advances to engage endoanchor 226A. After engagement, applier catheter 214, including endoanchor fixture 236 with endoanchor 226A, is withdrawn from endoanchor cartridge 202. As shown in FIG. 2H, multi-faceted flap 230A is pushed outward by endoanchor 226A as it is being withdrawn from endoanchor cartridge 202. In one or more embodiments, endoanchor 226A, applier catheter 214 and/or endoanchor fixture 236 may be labelled so that they can be observed under Flouro to help position endoanchor fixture 236 to engage endoanchor 226A. In embodiments having flaps to hold and/or segregate the endoanchors, the flaps may be flexible such that upon withdrawal of the applier catheter 214, the flaps may flex outward to allow the applier catheter and loaded endoanchor to pass thereby. The flaps may be resilient such that they return to their previous shape to prevent any endoanchors above them from exiting the cartridge.

FIG. 2I depicts peripheral support systems 238A, 238B, 238C, and 238N secured to endoanchor cartridge 202 for releasably securing endoanchors 226A, 226B, 226C, and 226N, respectively. Following is a further description of peripheral support system 238A for releasably securing endoanchor 226A. This description may be applied to the other peripheral support systems. Peripheral support system includes first upper support 240A, second upper support 240B, first lower support 240C, and second lower support 240D. As shown in FIG. 2I, first upper support 240A and second upper support 240B are secured to first and second coils, respectively, of endoanchor 226A. As shown in FIG. 2I, first lower support 240C and second lower support 240D are secured to the cross bar and a coil, respectively, of endoanchor 226A.

FIG. 2J is a magnified, isolated view of endoanchor 226A and second lower support 240D. Following is a further description of second lower support 240D and its connection to endoanchor 226A. This description may be applied to the other supports. Second lower support 240D includes endoanchor connecting portion 242A, area of weakness 242B, and cartridge connecting portion 242C. Endoanchor connecting portion 242A is secured to endoanchor 226A. Cartridge connecting portion 242C is secured to the inner surface of endoanchor cartridge 202. Area of weakness 242B connects endoanchor connecting portion 242A and cartridge connecting portion 242C. Applier catheter 214 may be configured to connect to proximal end of endoanchor 226A. Once applier catheter 214 is connected to endoanchor 226A, applier catheter 214 may be rotated or twisted (e.g., through manipulation of the handle of endoanchor delivery system 200), thereby breaking the area of weakness of first upper support 240A, second upper support 240B, first lower support 240C, and second lower support 240D to disconnect endoanchor 226A from the inner surface of endoanchor cartridge 202. Applier catheter 214 may include an endoanchor fixture (e.g., a D-shaped member) to connect applier catheter 214 to endoanchor 226A. The upper and lower supports may be formed of a polymeric material (e.g., a plastic material).

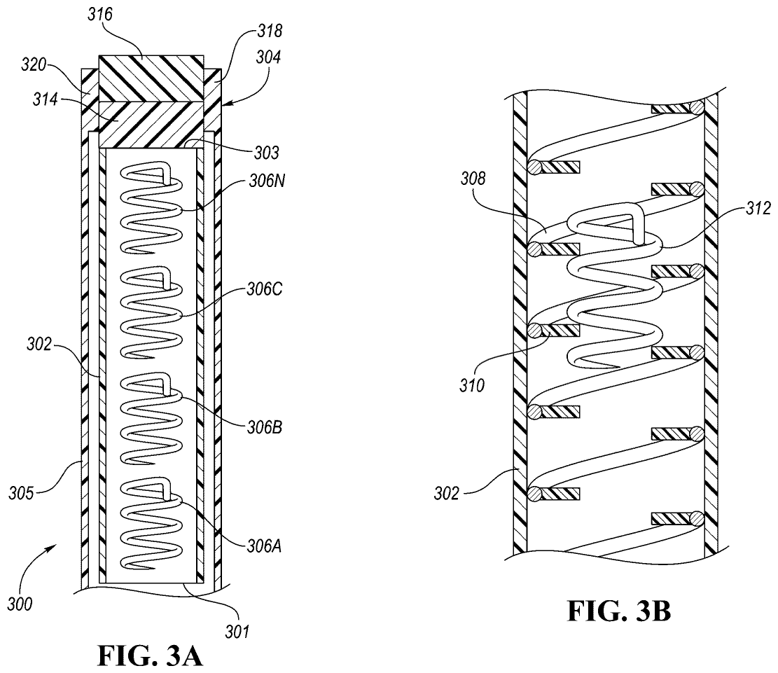

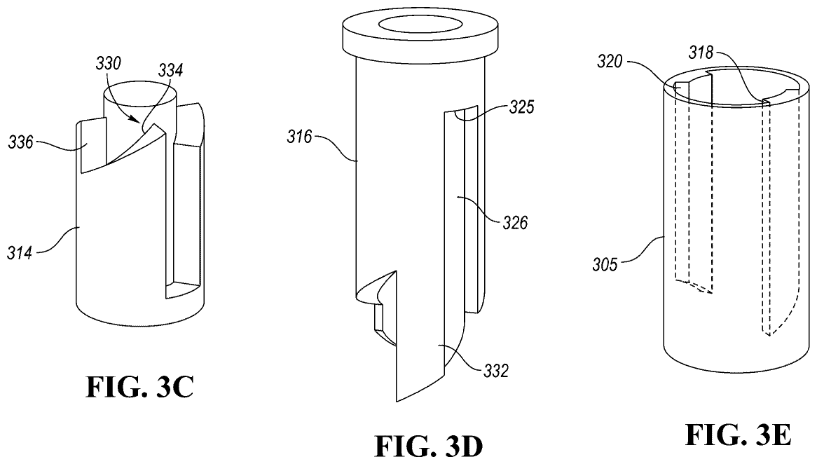

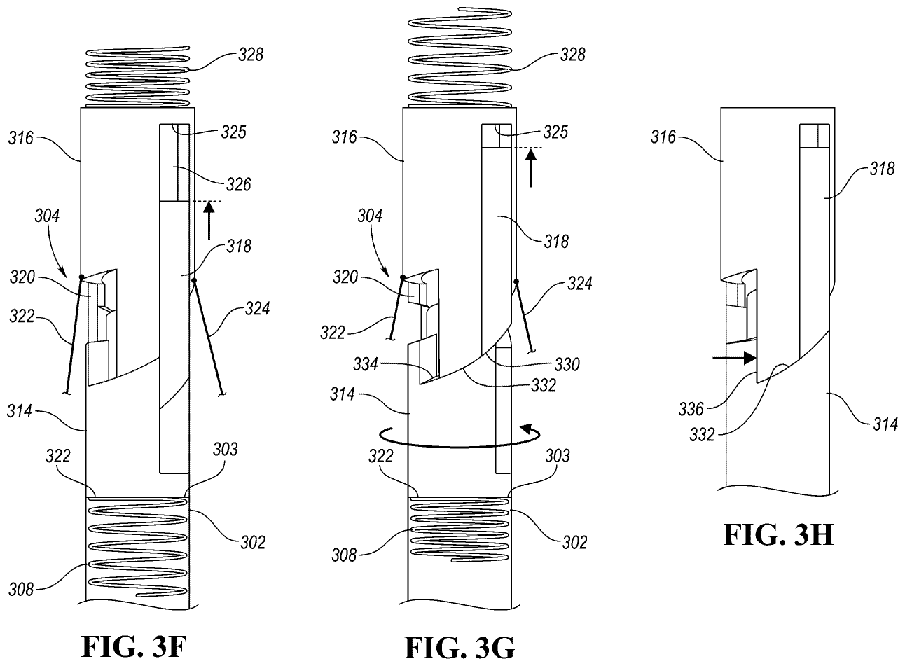

FIG. 3A depicts a schematic, cross section view of a portion of endoanchor delivery system 300 including endoanchor cartridge 302 and endoanchor dispensing system 304. Endoanchor cartridge 302 includes proximal end 301 and distal end 303. Endoanchor dispenser 304 is secured to distal end 303 of endoanchor cartridge 302. Proximal end 301 of endoanchor cartridge 302 may define an opening to load the endoanchors onto an applier catheter.

Endoanchor cartridge 302 is configured to receive, contain, and dispense a series of endoanchors 306A through 306N. Endoanchor dispenser 304 is configured to dispense endoanchors 306A through 306N (e.g., dispensing the endoanchors one by one). While FIG. 3A depicts four (4) endoanchors housed within endoanchor cartridge 302, the length of the cartridge may be modified to house more endoanchors (e.g., 2, 3, 4, 5, 6, 7, 8, 9, 10, 11, 12, 13, 14, 15, 16, 17, 18, 19, and 20 or any range of these two values).

FIG. 3B depicts a schematic, cross section view of endoanchor cartridge 302 including helical track 308 and protrusions 310 extending from helical track 308. Helical track 308 may contact the inner surface of endoanchor cartridge 302. Helical track 308 may be formed of a spring made of a metal material. FIG. 3B shows helical endoanchor 312, which may be one of endoanchors 306A through 306N. Protrusions 310 may be configured to retain endoanchor 312. As further described below, endoanchor dispenser 304 may be configured to rotate helical track 308 while not advancing the axially position of helical track 308, thereby urging endoanchor 312 to axially translate along protrusions 310 from a distal position to a proximal position for loading to an applier catheter (not shown) of endoanchor delivery system 300. Although only four (4) protrusions are shown, protrusions 310 may be secured to each coil along helical track 308.

Endoanchor dispenser 304 includes cam body 314, plunger 316, first stop member 318, and second stop member 320. FIGS. 3C, 3D, and 3E depict perspective views of cam body 314, plunger 316, and first and second stop members 318, respectively. In one or more embodiments, cam body 314 is secured to distal end 322 of helical track 308 and distal end 303 of endoanchor cartridge 302, as shown in FIGS. 3F and 3G. Cam body 314 is configured to rotate and axially translate distally and proximally at least partially within the endoanchor dispenser (e.g., relative to a post secured to endoanchor dispenser 304). Plunger 316 is configured to axially translate distally and proximally at least partially within the endoanchor dispenser (e.g., relative to a post secured to endoanchor dispenser 304). In one or more embodiments, plunger 316 is anti-rotational (e.g., it does not rotate). In one or more embodiments, first stop member 318 and second stop member 320 are fixed to sheath 305 of endoanchor dispenser 304.

Endoanchor dispensing system 304 is configured to transition from a retracted position where endoanchor 312 is retracted within endoanchor cartridge 302 and a loaded position where endoanchor 312 is loaded on an applier catheter (e.g., applier catheter 214).

FIG. 3F depicts a schematic side view of endoanchor dispenser 304 in a first deployment position between the retracted position and the loaded position. Distal stop 325 of channel 326 of plunger 316 is axially spaced apart from first stop member 318 in the retracted position. Plunger 316 axially translates in a proximal direction while transitioning into the deployment position as shown in FIG. 3F where first stop member 318 moves toward distal end 325 of channel 326. First wire 322 and second wire 324 are fixed to plunger 316 and configured to be pulled in a proximal direction (e.g., by a handle of endoanchor delivery system 300) to affect an axial translation of plunger 316. In one or more embodiments, spring 328 is configured to compress to cause an axial spring force to move plunger 316 axially in a proximal direction. Distal end of spring 328 may be secured to endoanchor dispenser 304.

FIG. 3G depicts a schematic side view of endoanchor dispenser 304 in a second deployment position moving toward the loaded position. In the second deployment position, distal end 330 of cam body 314 drops below first stop member 318. In this position, a spring (which may be helical track 308 or a spring secured to helical track 308) compresses within endoanchor cartridge 302, thereby causing axial spring force to translate cam body 314 axially in the distal direction. This axial upward movement causes ramp 334 of cam body 314 to contact finger 332 of plunger 316 and finger slides along ramp 334, thereby causing rotational movement of cam body 314. The rotational movement of cam body 314 rotates helical track 308, which is fixed thereto, to urge endoanchor 312 to axially translate from a distal position to a proximal position for loading to an applier catheter (not shown) of endoanchor delivery system 300.

FIG. 3H depicts endoanchor dispenser 304 in the loaded position. In the loaded position, finger 332 of plunger 316 contacts stop 336 of cam body 314 to stop rotational movement of cam body 314. In one or more embodiments, plunger 316 is axially raised, causing further rotational movement of finger 332 until it stops at stop 336. In this position, the endoanchor may be loaded in the applier catheter. The plunger 316 and cam body 314 may be locked in place to each other until a subsequent loading sequence.

In one or more embodiments, cam body 314 has 180 degree rotational symmetry such that the steps of loading an endoanchor may be repeated twice to cause a full 360 degree rotation of cam body 314 such that the process may be repeated to load the endoanchors (e.g., all the endoanchors) occupying the helical track 308.

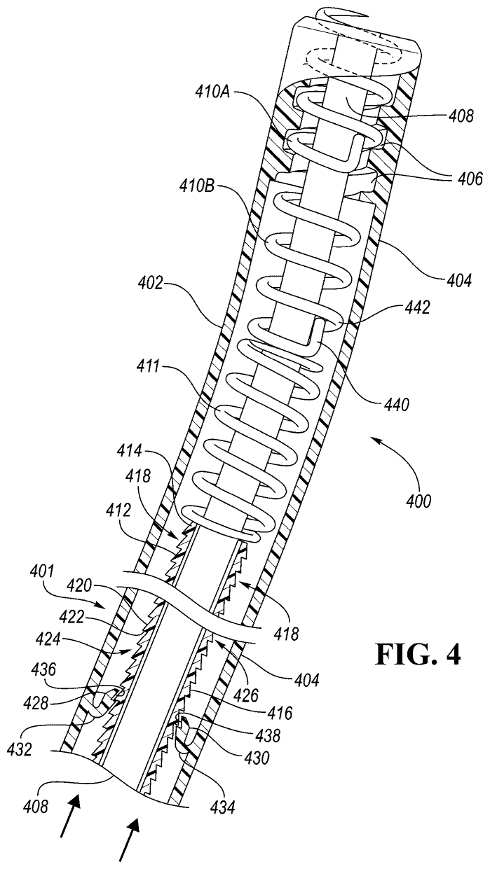

FIG. 4 depicts a cutaway side view of distal section 400 and proximal section 401 of endoanchor applier 402. Endoanchor applier 402 includes catheter 404 extending along the longitudinal axis of endoanchor applier 402 and internal threading 406 (e.g., helical threading) extending from inner surface of catheter 404 at a distal portion thereof. Endoanchor applier 402 includes driver 408 extending along the longitudinal axis of endoanchor applier 402 within catheter 404. Driver 408 may be rotationally driven to deliver multiple endoanchors (e.g., first endoanchor 410A, second endoanchor 410B, third endoanchor 410C, etc.) from distal section 400 of endoanchor applier 402.

Catheter 404 is configured to carry multiple endoanchors (e.g., first endoanchor 410A, second endoanchor 410B, third endoanchor 410C, etc.). While FIG. 4 depicts three endoanchors housed within catheter 404, catheter 404 may house more endoanchors (e.g., 4, 5, 6, 7, 8, 9, 10, 11, 12, 13, 14, 15, 16, 17, 18, 19, and 20 or any range of these two values).

Endoanchor applier 402 includes lift tube 412 having distal section 414 and proximal section 416. As depicted in FIG. 4, lift tube 412 includes serrations 418 facing catheter 404. Each serration 418 includes ramp 420 and stop 422. Each ramp 420 may incline from a distal location to a proximal location. Stop 422 may extend perpendicular to the inner surface of lift tube 412. As depicted in FIG. 4, serrations 418 extend along first path 424 and second path 426 of catheter 404 such that first path 424 opposes second path 426 along the outer surface of catheter 404 (e.g., first path 424 and second path 426 are spaced apart from each other by 180 degrees). In other embodiments, catheter 404 may include additional serration paths (e.g., 3 or 4 serration paths) that are equally spaced apart from each other or non-equally spaced apart from each other along the outer surface of catheter 404. The serration paths may be parallel to each other. Each of the paths may have a radial width (e.g., each of the serration paths only extend radially within the radial width). In another embodiment, a single serration path may extend around an entire circumference of the life tube 412 and may extend a length of lift tube 412 (e.g., a portion of the length or the entire length of lift tube 412).

Serrations 418 of first path 424 are configured to mate to tongue 428 and serrations 418 of second path 426 are configured to mate to tongue 430. Tongue 428 extends from tongue arm 432 extending from inner surface of proximal section 401 of catheter 404. Tongue 430 extends from tongue arm 434 extending from inner surface of proximal section 401 of catheter 404. Tongue 428 includes groove 436 configured to engage serrations 418 of first path 424. Tongue 430 includes groove 438 configured to engage serrations 418 of second path 426. As shown in FIG. 4, tongue 428 and tongue 430 are longitudinally aligned with each other. In other embodiments, tongue 428 and tongue 430 may be longitudinally offset each other. In another embodiment, there may be a single tongue that extends circumferentially around an entire circumference of the lift tube and extends the length of the lift tube. In one or more embodiments, serrations 418 may only extend within a proximal region of lift tube 412 (e.g., a distal portion of lift tube 412 does not include the serrations).

Lift tube 412 may be advanced longitudinally in a distal direction using an advancement mechanism (e.g., translation of a slider, actuation of a push button, or turning of a knob). The advancement mechanism may be disposed in a handle (not shown) of endoanchor applier 402. In one or more embodiments, the advancement mechanism translates distally one endoanchor length with each actuation. In other embodiments, the advancement mechanism may translate one serration each actuation or a multiple of one serration (e.g., 2 or 3 serrations) each actuation.

Ramps 420 of serrations 418 of lift tube 412 move along tongues 428 and 430 in a first direction. Tongues 428 and 430 are prevented from travelling along a second direction opposite the first direction by stops 422 of serrations 418, thereby maintaining the longitudinal positioning of lift tube 412. Tongues 428 and 430 may be located in any longitudinal portion of the applier proximal to the point of engagement with the most proximal endoanchor. In one embodiment, they may be located within the proximal section 401. In another embodiment, they may be located in a portion of the catheter that is within the handle.

Rotation of driver 408 coupled with the distal directional force imparted by lift tube 412 advances the next endoanchor into internal threading 406 of distal section 400 of endoanchor applier 402. As shown in FIG. 4, the directional force imparted by lift tube 412 acts on spring 411, which in turn, acts on the endoanchors to move the distalmost endoanchor into internal threading 406. In other embodiments, lift tube 412 directly acts on the endoanchors to move the distalmost endoanchor into internal threading 406. FIG. 4 depicts driver 408 extending within the cavities formed by first endoanchor 410A and second endoanchor 410B. In one or more embodiments, when an endoanchor is to be loaded into a deployment position, a clinician actuates the actuator device to rotate driver 408. The rotation of driver 408 combined with the force provided by the longitudinal advancement of lift tube 412 feeds an endoanchor into internal threading 406. The subsequent actuation of the actuator deploys the endoanchor within internal threading 406 and feeds the next distalmost endoanchor into internal threading 406. In one or more embodiments, the internal threading has a distal threading length, and the second endoanchor has a second endoanchor length complimentary to the distal threading length (e.g., the same or substantially the same axial length).

Endoanchor 410B includes crossbar 440 extending from main body 442 of endoanchor 410B. Crossbar 440 may be provided with strength to withstand a torque applied by driver 408 to implant the endoanchor through a prosthesis and a blood vessel wall. In one or more embodiments, driver 408 may include a “D” shaped elongated shaft with a planar face connected to a semi-circular surface. Driver 408 is shaped to fit within the inner diameters of the endoanchors with the planar face engaging the crossbar of each endoanchor such that torque is transferred from driver 408 to the endoanchors. In one or more embodiments, while the coupled action of driver rotation and lift tube translation advances the next endoanchor into the internal threading, only the driver rotation is used to implant the endoanchor through a prosthesis and a blood vessel wall.

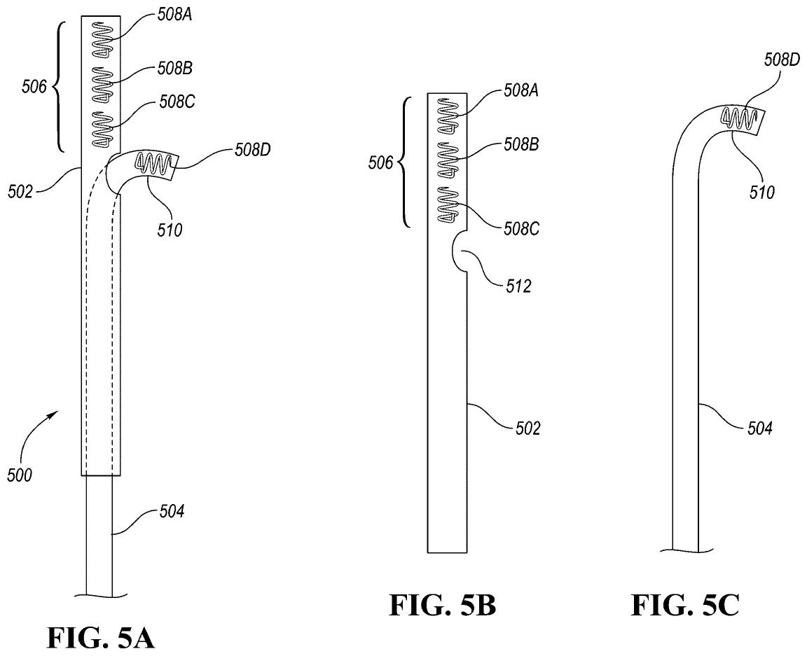

FIG. 5A depicts a side, schematic view of endoanchor delivery system 500 including sheath 502 and applier catheter 504 extending within sheath 502. FIG. 5B depicts a side, schematic, isolated view of sheath 502. FIG. 5C depicts a side, schematic, isolated view of applier catheter 504.

Sheath 502 includes distal section 506 configured to preload first endoanchor 508A, second endoanchor 508B, and third endoanchor 508C. The endoanchors may be preloaded distally through a distal opening defined in distal section 506 of sheath 502. After the endoanchors are preloaded distally within distal section 506, sheath 502 is tracked through the vasculature of a patient. The preloaded endoanchors may be maintained in their position within distal section 506 of sheath 502 by internal threading or by flaps or other mechanisms as described above in FIG. 2. In one or more embodiments, sheath 502 has a generally straight shape but has flexibility to permit it to track through blood vessels (e.g., iliofemoral vessel).

As shown in FIGS. 5A and 5C, fourth endoanchor 508D is loaded into distal section 510 of applier catheter 504. After fourth endoanchor 508D is preloaded distally within distal section 510 of applier catheter 504, applier catheter 504 is tracked through sheath 502, which was previously tracked through the vasculature of the patient. Sheath 502 defines an opening 512 in the sidewall thereof. Distal section 510 of applier catheter 504 carrying endoanchor 508D may be steered or deflected through opening 512 of sheath 502 to deliver endoanchor 508D. After delivery of endoanchor 508D, applier catheter 504 is retracted inside of sheath 502. Distal section 510 of applier catheter 504 is configured to load endoanchor 508C. The steering, delivering, and loading steps may be repeated to orient distal section 510 of applier catheter 504 around the circumference of a blood vessel wall to secure an implant thereto. The diameter of applier catheter 504 may be any of the following sizes or in the range of any two of the following sizes: 12, 13, 14, 15, 16, 17, 18, 19, 20, 21, 22, 23, and 24 to accommodate steering angles in the range of 0 to 120 degrees. In one or more embodiments, applier catheter 504 may be steered to permit application of the endoanchors to acute angle anatomies. Applier catheter 504 is configured to straighten or be straightened when retracted inside sheath 502 to facilitate loading of another endoanchor. Applier catheter 504 may be deflectable via one or more wires extending along a length thereof that may be tensioned at the handle to cause deflection in the desired direction. Alternatively, applier catheter may be formed with a pre-determined bend at its distal end (e.g., of about 90 degrees) but may be held straight by the sheath 502 when therein or by a stiff wire. In this embodiment, the applier may extend out of the opening 512 naturally when aligned therewith due to predisposition to a bent shape. To straighten the applier when in the sheath, a stiff wire may be extended within a longitudinal lumen thereof to force it into a straight configuration. A new endoanchor may then be loaded and the stiff wire may be retracted to allow the tip to bend again to exit the opening 512.

In one or more embodiments, the endoanchor(s) may include a radiopaque marking material (e.g., gold, platinum, iridium, etc.) configured to be visualized under fluoroscopy. The use of a radiopaque marking material is configured to aid the clinician in deploying each endoanchor in an appropriate location. In at least one embodiment, the endoanchor(s) may have one or more radiopaque markers at their tip or leading edge (e.g., tip of a helical anchor) to assist with visualization of the endoanchor as it is being positioned and/or inserted. In another embodiment, the endoanchor(s) may alternatively or additionally have one or more radiopaque markers on their base or the trailing edge to assist with visualization of endoanchor during and/or after deployment. The radiopaque markers may have a shape that is asymmetrical on at least one axis in a plane visible under fluoroscopy to assist with orientation. For example, a letter “E” or letter “C” may be used, as this allows the physician to know whether the marker is facing forwards or backwards.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An endoanchor delivery system comprising:
an endoanchor sheath defining an endoanchor sheath lumen, the endoanchor sheath having a proximal portion and a distal portion, the endoanchor sheath defining a side opening within the distal portion of the endoanchor sheath;
a series of endoanchors loaded into the lumen of the endoanchor sheath at the distal portion thereof and disposed along a longitudinal axis of the endoanchor sheath in a loaded state, the series of endoanchors includes a first endoanchor and a second endoanchor, the first endoanchor is located inward the second endoanchor along the longitudinal axis of the endoanchor sheath; and
an applier catheter extendible within the endoanchor sheath, the applier catheter having a proximal portion and a distal portion, the applier catheter is configured to load the first endoanchor at the distal portion of the applier catheter and to extend the distal portion of the applier catheter through the side opening to deploy the first endoanchor to anchor an implant to a blood vessel wall.

2. The endoanchor delivery system of claim 1 further comprising an endoanchor cartridge located in the distal portion of the endoanchor sheath and configured to carry the series of endoanchors.

3. The endoanchor delivery system of claim 2, wherein the endoanchor cartridge includes a first flap secured proximal the first endoanchor and a second flap secured proximal the second endoanchor, the first flap has a holding state to hold the first endoanchor within the endoanchor cartridge and a loading state to load the first endoanchor to the applier catheter, and the second flap has a holding state to hold the second endoanchor within the endoanchor cartridge and a loading state to load the first endoanchor to the applier catheter.

4. The endoanchor delivery system of claim 2, wherein the endoanchor cartridge includes a first support system for releasably securing the first endoanchor and a second support system for releasably securing the second endoanchor.

5. The endoanchor delivery system of claim 4, wherein the first support system includes one or more areas of weakness and the second support system includes one or more areas of weakness.

6. The endoanchor delivery system of claim 1, wherein the endoanchor sheath includes an inner surface and an outer surface, and further comprising a biasing element secured to the inner surface of the sheath, the biasing element at least partially aligning with the side opening.

7. The endoanchor delivery system of claim 6, wherein the biasing element includes a stretched state during loading of the first endoanchor by the applier catheter and an unstretched state when deploying the first endoanchor through the side opening.

8. The endoanchor delivery system of claim 6, wherein the biasing element includes a distal base, a proximal base, and a middle portion extending therebetween, the distal base fixedly secured to the inner surface of the endoanchor sheath, the proximal base slidably secured to the inner surface of the sheath.

9. The endoanchor delivery system of claim 7, wherein the middle portion bows outward in the unstretched state to create a stop to deflect the applier catheter through the side opening.

10. The endoanchor delivery system of claim 1 further comprising an endoanchor fixture extending from the distal portion of the applier catheter an extending length corresponding to a length of the first endoanchor.

11. The endoanchor delivery system of claim 10 further comprising an endoanchor cartridge located in the distal portion of the endoanchor sheath and configured to carry the series of endoanchors, wherein the endoanchor fixture is configured to releasably hold the first endoanchor to withdraw it proximally from the endoanchor cartridge.

12. An endoanchor delivery system comprising:
an endoanchor sheath having a proximal portion terminating in a proximal end and a distal portion terminating in a distal end, the endoanchor sheath defining an endoanchor sheath lumen, the endoanchor sheath defining a side opening within the distal portion of the endoanchor sheath;
an endoanchor cartridge located within the distal portion of the endoanchor sheath and extending along a longitudinal axis;
a series of endoanchors loaded into the endoanchor cartridge and disposed along the longitudinal axis of the endoanchor cartridge in a loaded state, the series of endoanchors includes a first endoanchor and a second endoanchor, the first endoanchor is located inward the second endoanchor along the longitudinal axis of the endoanchor cartridge;
an applier catheter extendible within the endoanchor sheath, the applier catheter having a proximal portion and a distal portion; and
an endoanchor dispenser extending from the distal end of the endoanchor sheath, the endoanchor dispenser is configured to be actuated to load the first endoanchor at the distal portion of the applier catheter,
the applier catheter is configured to extend the distal portion of the applier catheter through the side opening to deploy the first endoanchor to anchor an implant to a blood vessel wall.

13. The endoanchor delivery system of claim 12, wherein the endoanchor cartridge includes a helical track to retain the first endoanchor and the second endoanchor, the endoanchor dispenser is configured to rotate the helical track to urge the first endoanchor to axially translate from a distal position to a proximal position within the endoanchor cartridge to load the first endoanchor to the applier catheter.

14. The endoanchor delivery system of claim 12, wherein the endoanchor dispenser includes a cam body, a plunger, and a stop member collectively configured to load the first endoanchor to the applier catheter.

15. The endoanchor delivery system of claim 14, wherein the stop member is fixed to the endoanchor sheath.

16. The endoanchor delivery system of claim 14, wherein the cam body is configured to rotate and axially translate distally and proximally at least partially within the endoanchor dispenser, and the plunger is configured to axially translate distally and proximally at least partially within the endoanchor dispenser.

17. An endoanchor delivery system comprising:
a shaft extending to define a shaft lumen, the shaft has a proximal portion and a distal portion, the shaft includes an inner surface has an internal threading and an outer surface, the internal threading is adjacent a distal end of the shaft;
a series of endoanchors loaded into the shaft lumen at the distal portion thereof and disposed along a longitudinal axis of the shaft, the series of endoanchors includes a first endoanchor and a second endoanchor, the first endoanchor is located inward the second endoanchor along the longitudinal axis of the shaft;
a driver extending along the longitudinal axis of the shaft and through the inner diameters of the first and second endoanchors; and
a spring situated within the shaft, a combination of the spring imparting a spring force and the driver rotating collectively loads the second endoanchor into the internal threading.

18. The endoanchor delivery system of claim 17 further comprising an actuator configured to actuate the spring force and the driver rotation via a first actuation of the actuator, the first actuation is configured to translate the second endoanchor into the internal threading.

19. The endoanchor delivery system of claim 17, wherein the actuator configured to actuate the spring force and the driver rotation via a second actuation of the actuator, the second actuation is configured to translate the first endoanchor into the internal threading and the second endoanchor into a deployment position within an implant and a blood vessel wall.

20. The endoanchor delivery system of claim 17, wherein the internal threading has a distal threading length, the second endoanchor has a second endoanchor length complimentary to the distal threading length.
